(19) United States
(12) Patent Application Publication
(10) Pub. No.: US 2024/0009398 A1
(43) Pub. Date: Jan. 11, 2024

(54) SYSTEMS AND METHODS FOR BLOW-FILL-SEAL (BFS) ASSISTED DISPENSING AND DISABLEMENT

(71) Applicant: Koska Family Limited, East Sussex (GB)

(72) Inventors: Marc Andrew Koska, Dartmouth (GB); Max Hannon, London (GB)

(21) Appl. No.: 18/371,427

(22) Filed: Sep. 21, 2023

Related U.S. Application Data
(63) Continuation of application No. PCT/IB2022/052849, filed on Mar. 28, 2022.
(60) Provisional application No. 63/167,096, filed on Mar. 28, 2021.

(51) Int. Cl. A61M 5/28; A61M 5/50

(52) U.S. Cl. CPC A61M 5/282; A61M 5/50; A61M 2205/581; A61M 2209/04; A61M 2205/43; A61M 2205/273

(57) ABSTRACT

Systems and methods for disabling Blow-Fill-Seal (BFS) vials such as pre-filled vials containing a vaccine or other medicament via utilization of an assistive dispensing device to selectively compress the fluid reservoir of the BFS vial. The assistive dispensing device can aid in applying compressive force to assist in dispensing the liquid from the vial, and may also be utilized to lock in a mated position so that the BFS vial may not be reused and/or is otherwise readily identifiable as having already been administered.

### CROSS-REFERENCE TO RELATED APPLICATIONS

This application is a Continuation Application of PCT/162022/052849 filed on Mar. 28, 2022 in the name of Koska et al. and titled SYSTEMS AND METHODS FOR BLOW-FILL-SEAL (BFS) ASSISTED DISPENSING AND DISABLEMENT, which PCT Application claims benefit and priority under 35 U.S.C. § 119(e) to, and is a Non-provisional of, U.S. Provisional Patent Application No. 63/167,096 filed on Mar. 28, 2021 and titled “SYSTEMS AND METHODS FOR BLOW-FILL-SEAL (BFS) VIAL DISPENSING AND DISABLEMENT.” Each of these Applications is hereby incorporated by reference herein in its entirety and for all purposes.

### TECHNICAL FIELD

The present disclosure describes systems, methods and devices for assisted dispensing and disablement of Blow-Fill-Seal (BFS) pre-filled, single-dose medicament delivery systems. In accordance with some embodiments, an assisted dispensing and disabling device may be embodied as an adjustable accessory component (referred to in some embodiments as a “cover”) that may be utilized with a BFS medicament delivery system and function to assist with gripping of a BFS vial component of the BFS medicament delivery system and/or dispensing of the contents thereof; and in some embodiments may also be operable to disable the BFS vial to prevent re-use of the BFS delivery systems.

### SUMMARY OF VARIOUS EMBODIMENTS

Embodiments of the disclosed subject matter provide systems, assemblies, kits, devices and methods for assisting with the medical delivery of a fluid agent to a patient. Using a blow-fill-seal (BFS) fabrication technique, a BFS component (such as a vial) is pre-filled with a fluid (e.g., liquid) agent intended for administration to a patient. In some embodiments, the liquid agent is administered via a needle or cannula coupled to the BFS vial (e.g., via use of a connector component).

The BFS medical delivery systems developed by Applicant provides a cost-effective option for pre-filled, single-dose delivery of fluid agents (e.g., vaccines or medicaments). In some embodiments, the BFS vials comprising components of such medical delivery systems are pressure activated by a user gripping and compressing a squeezable reservoir to dispense the fluid agent. Example dosage sizes for many fluid agents dispensed through Applicant's BFS medical delivery systems can be quite small (e.g., between three-tenths of a milliliter (0.3-ml) and three milliliter (3.0-ml)) and the geometry of BFS vials containing such substances are accordingly small in size. Applicant has recognized that while BFS medical delivery systems described herein provide a low-cost, sterile, single-use injection, the small geometry size of the BFS vials compared to the human hand may prove challenging for some users during dispensing. For example, users with larger fingers, low grip strength, or mobility issues (e.g., arthritis) may find it difficult to readily achieve a sufficient grip on a small, pressure-activated BFS delivery system (or a portion thereof such as the squeezable reservoir). This may result in unsatisfactorily compressing of the BFS chamber or reservoir, and difficulty in dispensing the proper pre-filled dose as a result.

Applicant has further recognized that in some situations it may be advantageous to allow for administration of less than all of the contents of a single-dose BFS vial. For example, while a BFS vial of a given volume may store an appropriate volume/dose of a fluid agent for a first user (e.g., an adult or a person receiving a primary dose of a vaccine), this may not be an appropriate dose for a second user (e.g., a child, or a person getting a booster of the vaccine).

Applicant has still further recognized that while it may be difficult to reuse a single-dose BFS medical delivery system as described herein, additional measures may be undertaken to further minimize the ability of the single-dose medical delivery system from being re-used. This may be particularly relevant in embodiments in which less than all of the contents of a BFS vial are injected (e.g., in accordance with dose restriction embodiments described herein).

Applicant has recognized that it may be advantageous for some users to be provided with an accessory component that: (i) assists with dispensing of fluid agents from single-dose BFS medical delivery systems; (ii) allows for more dosing controls or customizations (e.g., such that a smaller dose can be dispensed for a child, from a vial that holds sufficient contents to provide an adult dose); and/or (iii) disables the BFS vial after use to minimize the risk of it being re-used (which can increase risk of infection to patients).

Embodiments described herein provide systems, methods, kids and/or devices for assisted dispensing and disabling of the BFS vials by providing an accessory device that may comprise an adjustable cover operable to aid with gripping and dispensing, which may also be used to disable the BFS vials to deter re-use by preventing the collapsible/squeezable reservoir from being reformed/reinflated and/or to prevent any contents left in the BFS vial from being reused for a second patient after a first portion of the contents is injected into a first user.

### BRIEF DESCRIPTION OF THE DRAWINGS

The figures depict embodiments for purposes of illustration only. The accompanying figures are not necessarily drawn to scale, emphasis instead being placed upon illustrating the principles disclosed herein. The figures are included to further the understanding of the various aspects and embodiments and are incorporated in and constitute a part of this specification, but are not intended as a definition of the limits of any particular embodiment. In the figures, each identical or nearly identical component that is illustrated in various figures is represented by a like numeral. For example, the same or similar elements throughout various embodiment may include the same two base reference numbers, preceded with a different numeral. For purposes of clarity, not every component may be labeled in every figure. One skilled in the art will readily recognize from the following description that alternative embodiments of the systems and methods illustrated herein may be employed without departing from the principles described herein, wherein:

FIG. 1A is a perspective view of an exemplary BFS delivery system including an assisted dispensing and/or disablement device in an unassembled state in accordance with the present disclosure;

FIG. 1B is a perspective view of the BFS device of FIG. 1A with the assisted dispensing and/or disablement device in an open, first assembled state;

FIG. 1C is a top view of the BFS device of FIG. 1B;

FIG. 1D is a perspective view of the BFS assisted dispensing device and/or disablement device of FIG. 1B in a closed second assembled state, prior to dispensing;

FIG. 1E is a perspective view of an exemplary BFS delivery system including an assisted dispensing and/or disablement device in an unassembled state in accordance with the present disclosure;

FIG. 1F is a perspective view of the BFS assisted dispensing device and/or disablement device of FIG. 1E in an open, first assembled state;

FIG. 1G is a perspective view of an exemplary BFS assisted dispensing and/or disablement device in accordance with the present disclosure;

FIG. 1H is a top view of the device of FIG. 1G;

FIG. 1I is a bottom view of the device of FIG. 1G;

FIG. 1J is a front view of the device of FIG. 1G;

FIG. 1K is a side view of the device of FIG. 1G; FIG. 2A is a perspective view of an exemplary BFS injection system including an assisted dispensing and/or disablement device in an unassembled state;

FIG. 2B is a perspective view of the BFS device of FIG. 2A with the assisted dispensing and/or disablement device in an open, first assembled state;

FIG. 2C is a perspective view of the BFS device of FIG. 2A with the assisted dispensing and/or disablement device in an open, first assembled state

FIG. 2D is a top view of the BFS device of FIG. 2C;

FIG. 2E is a perspective view of the BFS assisted dispensing device and/or disablement device of FIG. 2D in a closed second assembled state, prior to dispensing;

FIG. 2F is a top, partial cross-sectional view of FIG. 2E;

FIG. 2G is a perspective view of FIG. 2E exposing components of the injection assembly;

FIG. 2H is a perspective view of FIG. 2G with a user gripping the BFS assisted dispensing and/or disablement device and advancing the injection assembly toward an injection target;

FIG. 2I is a cross-sectional side view of FIG. 2I with fluid having been moved from the fluid reservoir;

FIG. 2J is a perspective view of FIG. H engaged with a target and the user gripping the BFS assisted dispensing and/or disablement device to further advance engagement/locking;

FIG. 2K is a top, partial cross-sectional view of FIG. 2J;

FIG. 2L is a cross-sectional side view of FIG. 2J with much of the fluid having been dispensed; and

FIG. 3 is a flowchart of a method according to some embodiments.

### DETAILED DESCRIPTION

### I. Introduction

Embodiments of the present disclosure provide systems and methods for assisted dispensing of pre-filled medical delivery assemblies, for example, medicaments from a BFS delivery system. An assisted vial dispensing device is disclosed that aids a user in gripping the body of the BFS delivery system, for example the collapsible fluid reservoir. The assisted vial dispensing device comprises a larger, slip-resistant surface area for gripping by the user than the body of the BFS delivery system, which is pressure-activated by the user squeezing during injection. In addition, the assisted vial dispensing device may be lockable over the fluid reservoir once dispensing is complete in order prevent reuse of the reservoir.

II. BFS Assisted Dispensing and/or Disablement Systems and Methods

The examples of the apparatus discussed herein are not limited in application to the details of construction and the arrangement of components set forth in the following description or illustrated in the accompanying drawings. It will be understood to one of skill in the art that the apparatus is capable of implementation in other embodiments and of being practiced or carried out in various ways. Examples of specific embodiments are provided herein for illustrative purposes only and are not intended to be limiting. Also, the phraseology and terminology used herein is for the purpose of description and should not be regarded as limiting. Any references to examples, embodiments, components, elements or acts of the apparatus herein referred to in the singular may also embrace embodiments including a plurality, and any references in plural to any embodiment, component, element or act herein may also embrace embodiments including only a singularity (or unitary structure). Likewise, components disclosed as a single element may be made from separate elements or devices. References in the singular or plural form are not intended to limit the presently disclosed apparatus, its components, acts, or elements. The use herein of “including,” “comprising,” “having,” “containing,” “involving,” and variations thereof is meant to encompass the items listed thereafter and equivalents thereof as well as additional items.

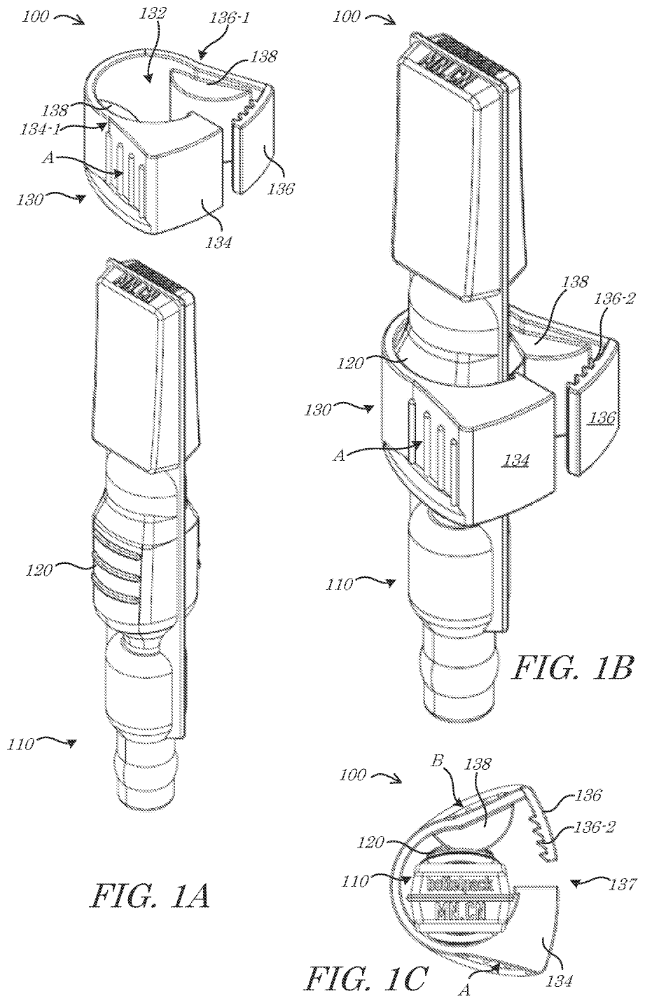

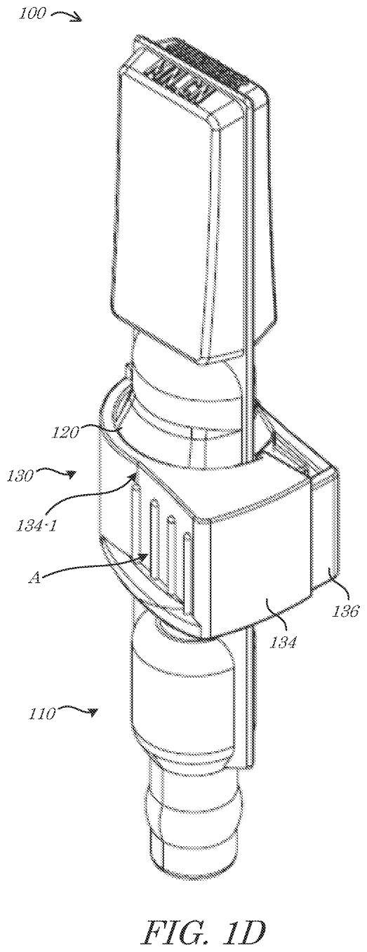

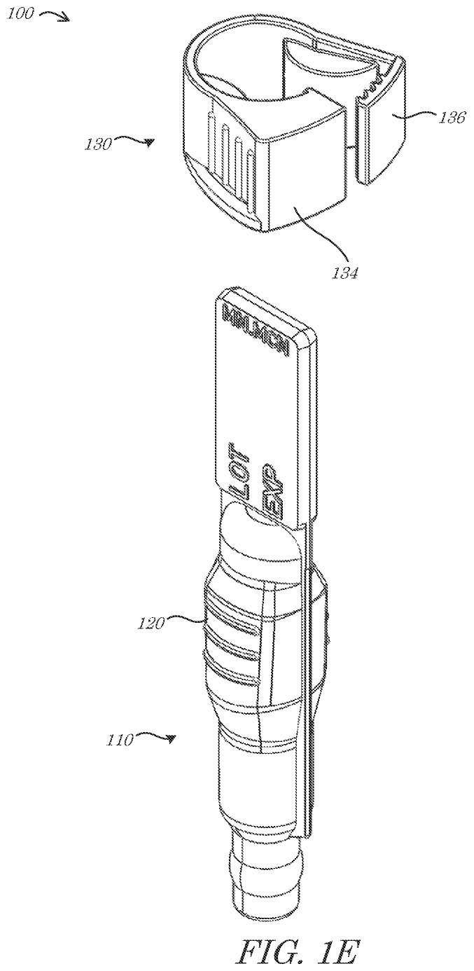

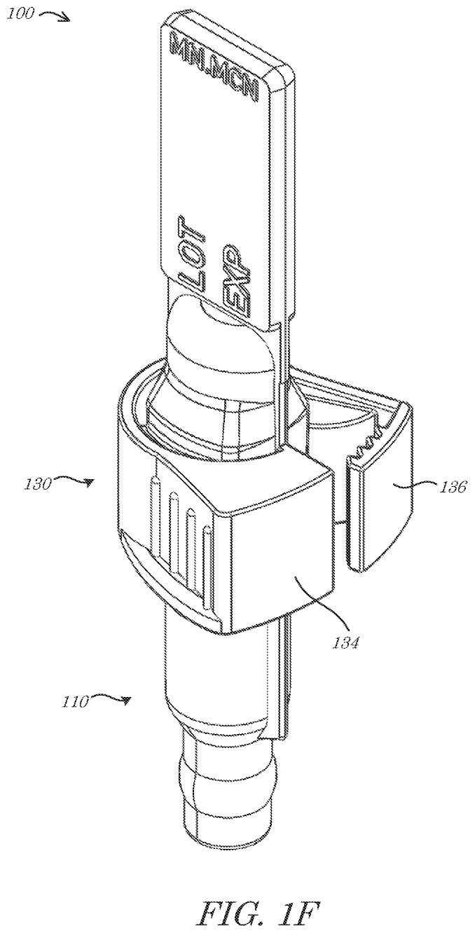

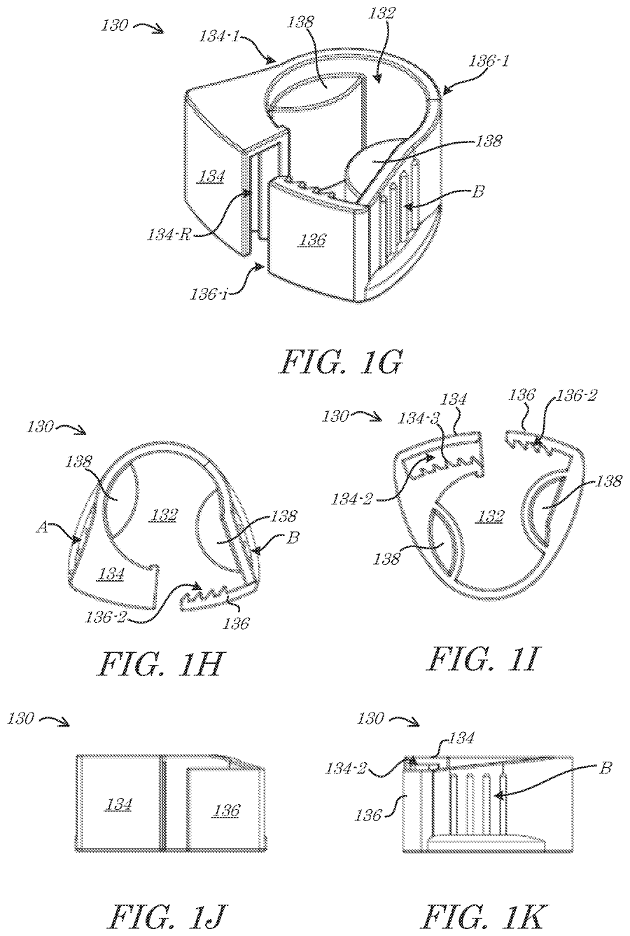

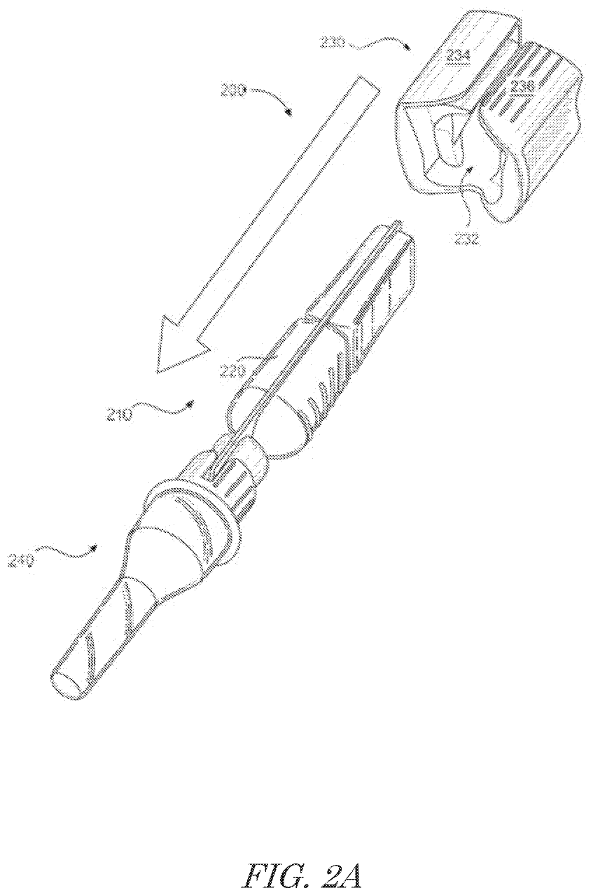

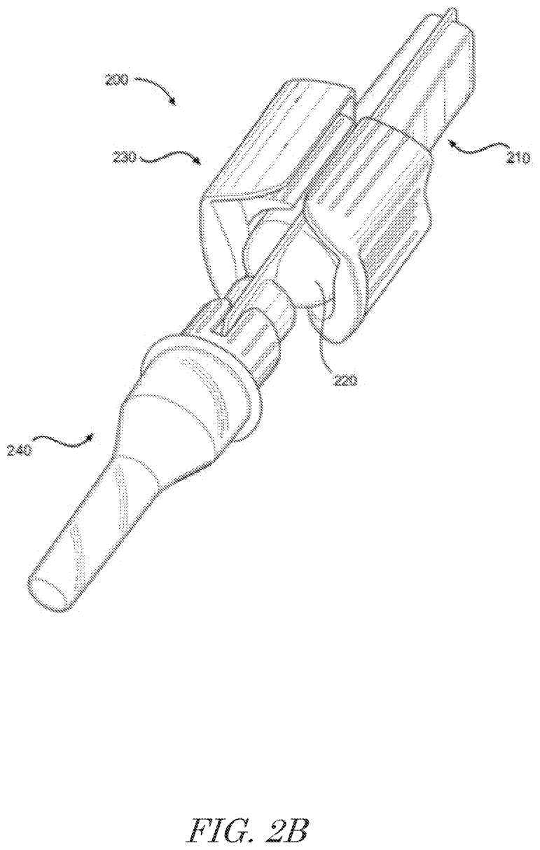

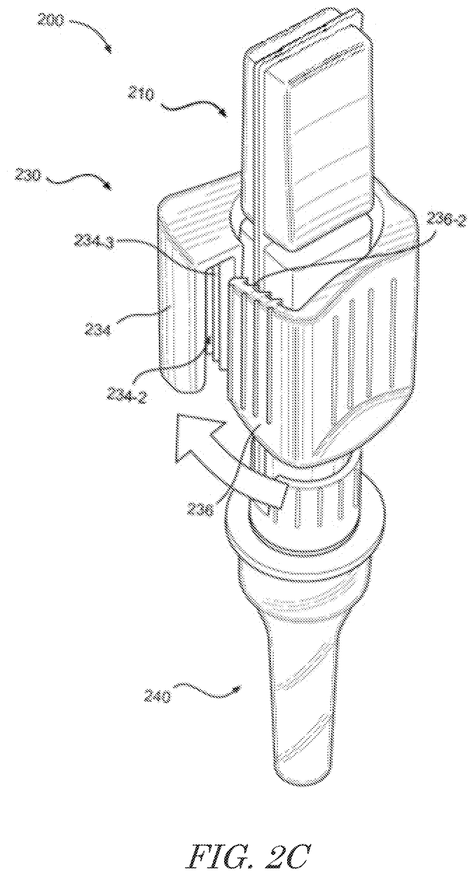

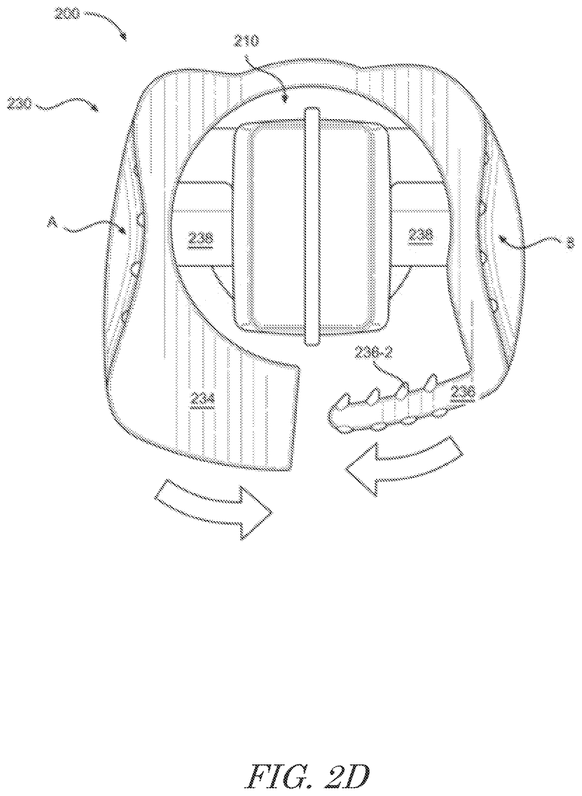

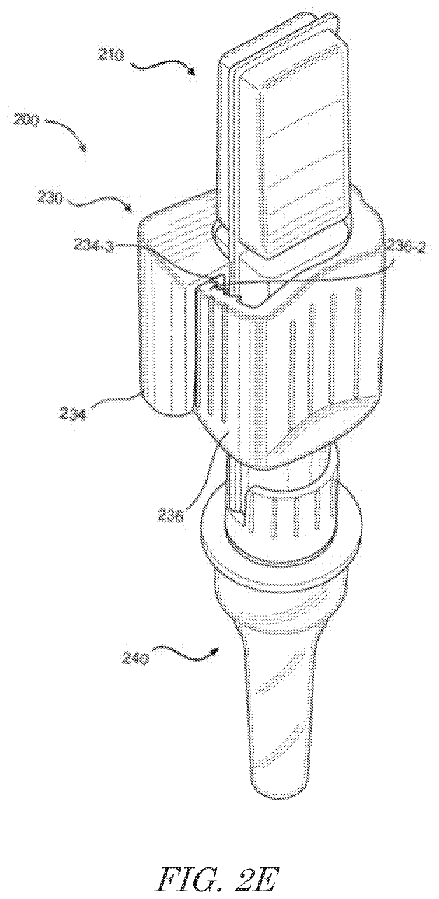

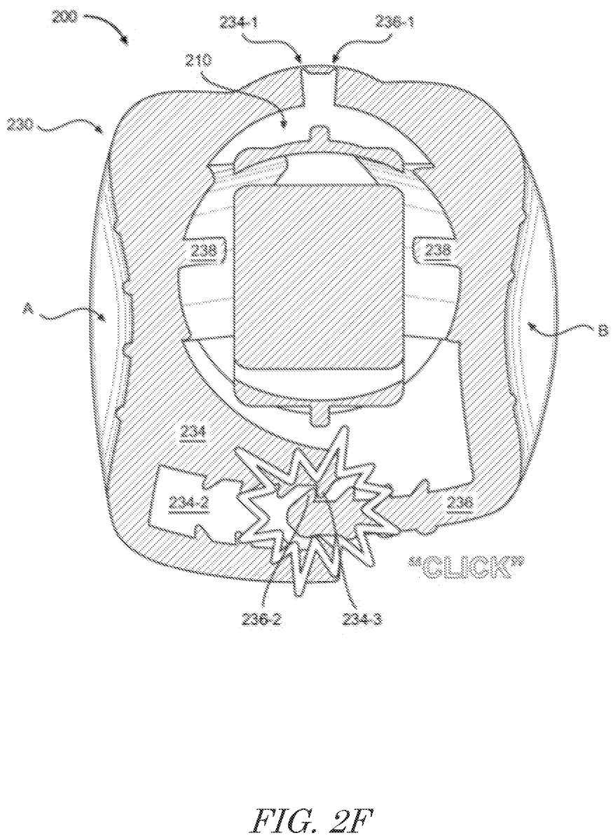

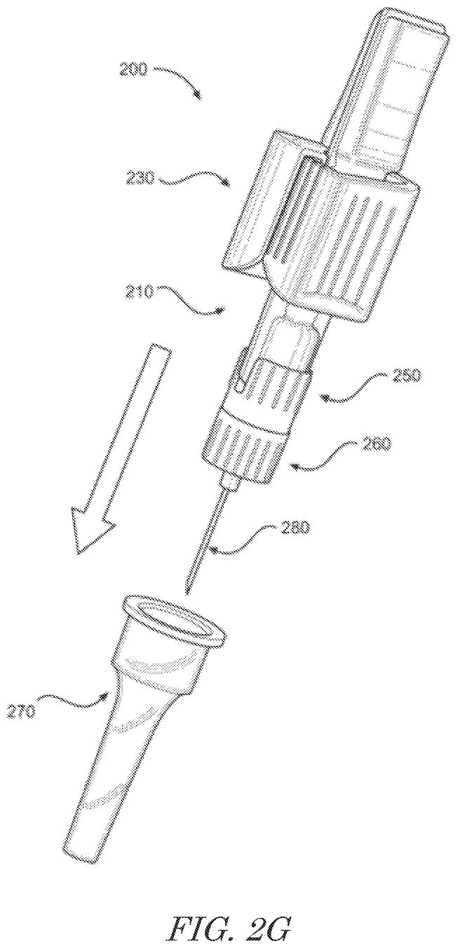

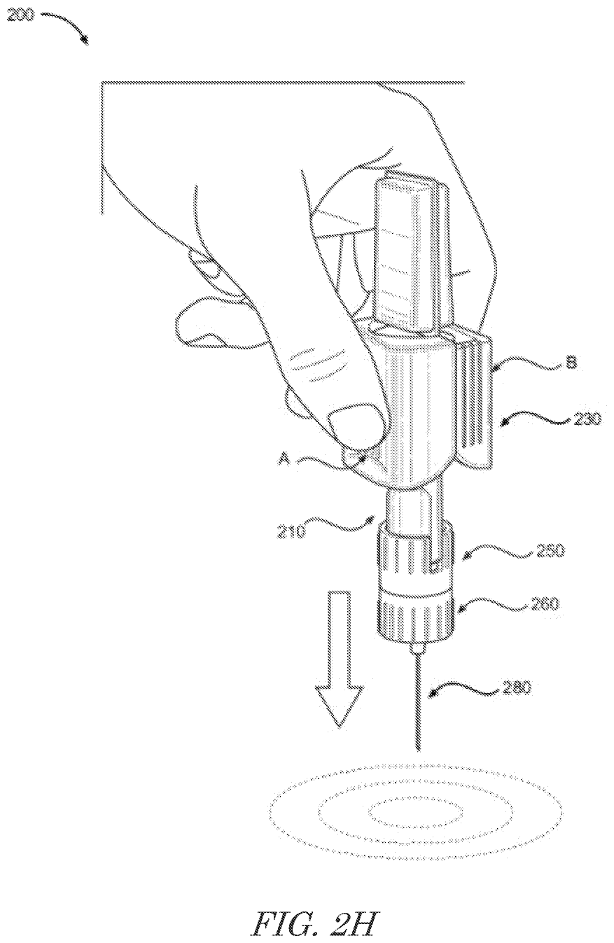

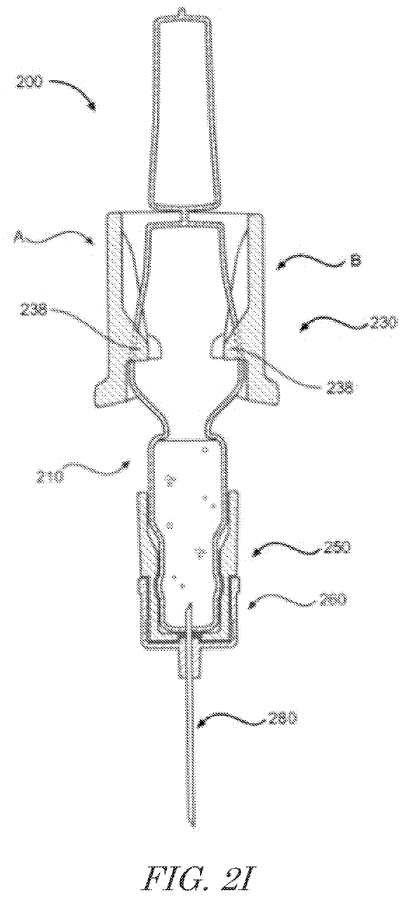

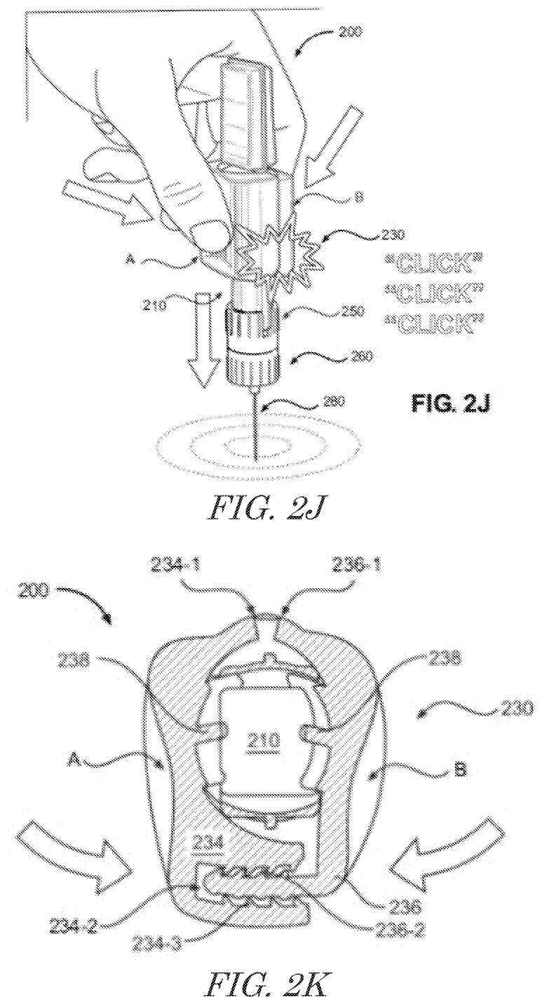

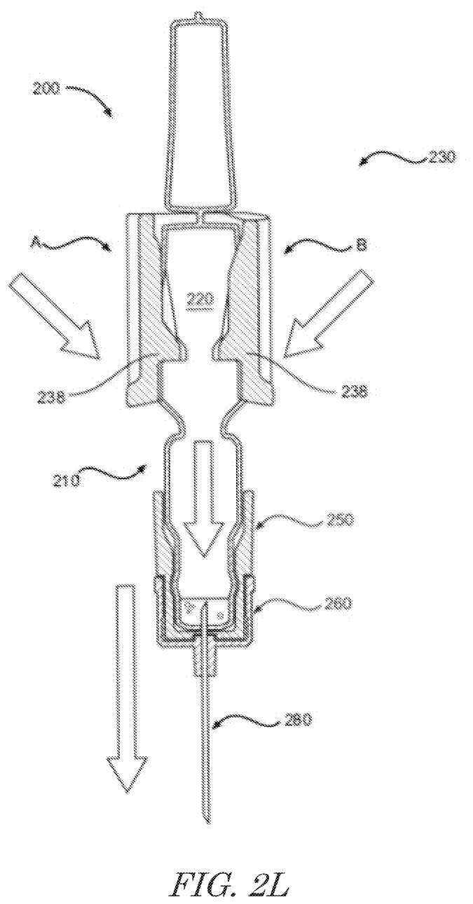

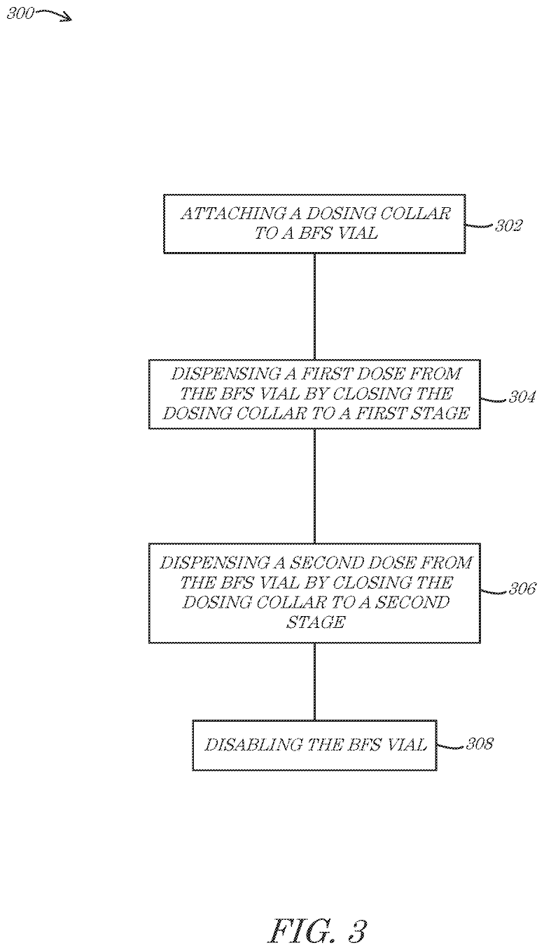

Referring initially to FIG. 1A, FIG. 1B, FIG. 1C, FIG. 1D, FIG. 1E, FIG. 1F, FIG. 1G, FIG. 1H, FIG. 1I, FIG. 1J, and FIG. 1K various views of a BFS delivery system 100 including an assisted delivery and/or disablement device according to an exemplary embodiment are shown. The BFS delivery system 100 may comprise, for example, a BFS bottle, container, or vial 110 comprising at least one collapsible fluid reservoir 120, e.g., as depicted in FIGS. 1A and 1E (as depicted, different configurations of the BFS vials 110 and/or the reservoirs 120 thereof may be utilized in the BFS delivery system 100). The fluid reservoir 120 may store, house, and/or be filled with one or more medicaments and/or desirable fluid substances such as saline solution, a vaccine, insulin, and/or other medical substances. According to some embodiments, the BFS system 100 may comprise a BFS assisted dispensing and/or disablement cover 130, for example a collar, sleeve, jacket, sheath or other device 130 defining an interior passage 132 sized to fit over or at least partially around fluid reservoir 120, e.g., as depicted in FIGS. 1B, 1C, and 1F in an open or first configuration. Assisted dispensing and/or disablement cover 130 may further comprise one or more of a first grip portion or surface “A” and a second grip portion or surface “B”, disposed on an outer surface of cover 130 that are sized and shaped to be gripped by a thumb and/or finger of the user as shown in FIG. 2H. Grip portions “A” and “B” may include a slip-resistant surface area, for example a textured surface, if so desired.

With continued reference to FIGS. 1A-1F, BFS assistive dispensing and/or disablement device 130 may be slid onto and/or wrapped at least partially around the fluid reservoir 120 of the BFS vial 110. When the BFS assistive dispensing and/or disablement device 130 is first slid over the BFS vial 110, the BFS assisted dispensing device 130 is initially in an open/first configuration, as illustrated in FIGS. 1B, 1C, and 1E, and may thereafter be selectively transitioned to a closed or second configuration. FIG. 1D illustrates the BFS delivery system 100 with the assisted dispensing and/or disablement device 130 in the second/closed configuration. In order to support the assistive dispensing device 130 on the vial 110, an adjustable closure 137 may be provided.

The BFS assistive dispensing and/or disablement device 130 may comprise, for example, an adjustable closure 137 comprising a first latch portion 134 (e.g., a first arm) that is operable to pivot or rotate about a first hinge 134-1, and in some embodiments may also include a second latch portion 136 (e.g., a second arm) that is operable to pivot or rotate about a second hinge 136-1. Hinges 134-1 and 136-1 may be, for example, a living hinge, or any other style hinge that allows for movement of the arms 134, 136. First latch portion 134 and second latch portion 136 may be any of a variety of latching mechanisms that when engaged are adjustable in order to selectively tighten the closure 137. In one exemplary embodiment, first latch portion or arm 134 may include a receiving end 134-R comprising a mating slot 134-2, within which are disposed one or more latching features 134-3, for example a plurality of indexing protrusions, teeth or catches, as depicted in FIG. 1G, FIG. 1H, and FIG. 1I. Arm 136 may include an insertion end 136-i comprising a plurality of indexing protrusions, catches or teeth 136-2 as also shown FIG. 1G, FIG. 1H, and FIG. 1I.

According to some embodiments, the latch arms 134, 136 may be operable, upon receiving a compressive force applied, e.g., via one or more of a first grip portion “A” and a second grip portion “B”, to advance and mate with each other. The second latch arm 136 may, for example, travel into the mating slot 134-2 and one or more of the plurality of protrusions, such as teeth 136-2 may engage with one or more cooperative protrusions, for example catches 134-3, coupling the second latch arm 136 to (e.g., inside of) the first latch arm 134; thereby closing the BFS assistive dispensing device 130 and/or bounding the interior passage 132 thereof.

In some embodiments, and as depicted in FIG. 1A, FIG. 1B and FIG. 1C, the BFS assistive dispensing and/or disablement device 130 may comprise one or more compression structures 138 disposed within the interior passage 132 such that engagement and/or mating of the latch arms 134, 136 causes the compression structure(s) 138 to exert inward radial force (e.g., compressive force) upon the fluid reservoir 120. In such a manner, for example, selective and/or progressive closing and/or tightening of the BFS assistive dispensing device 130 may exert increasing force upon the fluid reservoir 120, thereby increasing the pressure exerted upon the fluid(s) stored therein. According to some embodiments, such when the BFS vial 110 is selectively punctured (not shown) and/or the fluid reservoir 120 is otherwise in communication with the atmosphere, the pressure exerted by the tightening of the BFS assistive dispensing and/or disablement device 130 about the fluid reservoir 120 may cause the contents of the fluid reservoir 120 to be expelled therefrom.

According to some embodiments, the number of teeth 136-2 and corresponding catches 134-3 may be configured to define a plurality of stages of compression of the fluid reservoir 120. A first “click” that engages a first one of the teeth 136-2 with a first one of the catches 134-3, for example, may either simply secure the device 130 closed or may cause the compression structure 138 to compress the fluid reservoir 120 to a first degree. A second “click” that further engages the first one of the teeth 136-2 with a second one of the catches 134-3 and further causes a second one of the teeth 136-2 to engage with the first one of the catches 134-3, may cause the compression structure 138 to compress the fluid reservoir 120 to a second (e.g., higher) degree. In such a manner, the number and/or spacing of the respective teeth 136-2 and catches 134-3 may be configured to compress the fluid reservoir 120 in various stages, degrees, and/or amounts and may be readily varied, as desired. In some embodiments, the compression structure 138 may be varied in number, size, and/or geometry to achieve a desired level of compression of the fluid reservoir 120 from a variety of positions and/or angles. In some embodiments, the compression structure 138 may, for example, comprise a thicker portion of interior passage 132 and may not be distinct projection. Likewise, the respective teeth 136-2 and catches 134-3 may be configured with alternate geometries, shapes, sizes and/or angles as well.

In some exemplary embodiments, the mating of the latch arms 134, 136 may further be configured to prevent disengagement thereof, for example the catches 134-3 and teeth 136-2 may be angled in opposing directions to permit advancement of the second latch arm 134 into the mating slot 134-2 but not for removal thereof. This may be achieved in some embodiments, for example, by having angled or smoothed contact surfaces on a first side thereof, while a second side thereof may comprise contact surfaces that are configured to engage to prevent movement in the opposite direction, similar to a pawl and ratchet. In such a manner, once the fluid reservoir 120 has been compressed by the BFS assistive dispensing and/or disablement device 130, the BFS assistive dispensing device 130 may not be removable therefrom (e.g., without destruction of one or more of the engagement components thereof).

Fewer or more components 110, 120, 130, 132, 134, 134-1, 134-2, 134-3, 134-R, 136, 136-1, 136-2, 136-i, 137, 138 and/or various configurations of the depicted components 110, 120, 130, 132, 134, 134-1, 134-2, 134-3, 134-R, 136, 136-1, 136-2, 136-i, 137, 138 may be included in the BFS delivery system 100 without deviating from the scope of embodiments described herein. In some embodiments, the components 110, 120, 130, 132, 134, 134-1, 134-2, 134-3, 134-R, 136, 136-1, 136-2, 136-i, 137, 138 may be similar in configuration and/or functionality to similarly named and/or numbered components as described herein. In some embodiments, the BFS delivery system 100 may comprise a BFS medicament delivery system configured to execute, conduct, and/or facilitate methods described herein such as the method 300 of FIG. 3 herein, or portions thereof.

Referring now to FIG. 2A, FIG. 2B, FIG. 2C, FIG. 2D, FIG. 2E, FIG. 2F, FIG. 2G, FIG. 2H, FIG. 2I, FIG. 2J, FIG. 2K, and FIG. 2L, various views of a BFS injection system 200 according to some exemplary embodiments are shown. The BFS injection system 200 may comprise, as is depicted in the perspective view of FIG. 2A for example, a BFS vial 210 comprising a fluid reservoir 220, a BFS assistive dispensing and/or disablement device 230 (e.g., a “dosing collar”) defining an interior passage 232 into which the BFS vial 210 may be inserted, and an injection assembly 240 coupled to a distal end of the BFS vial 210.

In some embodiments, the BFS assistive dispensing device 230 may comprise a female latch arm 234 that is operable to pivot about a first hinge point 234-1 and/or that defines a latching slot 234-2 that comprises one or more latching features 234-3, as described above and illustrated in FIG. 2F. The BFS dispensing device 230 may also comprise a male latch arm 236 that is operable to pivot about a second hinge point 236-1 and/or that defines one or more exterior latching features 236-2 in some embodiments. One or more compression structures 238 may be disposed within and/or project radially into the interior passage 232, for example as shown in FIGS. 2D, 2F and 2K.

As depicted in FIG. 2A, the combined injection assembly 240 and BFS vial 210 may be received within interior passage 232 of the BFS assistive dispensing and/or disablement device 230 (e.g., as a first step). As depicted in the perspective view of FIG. 2B, the BFS dispensing and/or disablement device 230 may be positioned (e.g., in an open state or configuration) about and/or around the fluid reservoir 220 of the combined injection assembly 240 and BFS vial 210 during the first step. According to some embodiments, such as in accordance with a second step depicted in FIG. 2C and FIG. 2D, the BFS dispensing and/or disablement device 230 may be closed by application of pressure that urges the male latch arm 236 into the latching slot 234-2 of the female latch arm 234, thereby engaging the interior and exterior latching features 234-3 and 236-2.

As depicted in FIG. 2E and FIG. 2F, an audible and/or otherwise apparent engagement (for example a visible indication) of the interior and exterior latching features 234-3 and 236-2 may indicate a first level (or stage) of engagement and/or mating of the latch arms 234, 236 during the second step. The BFS dispensing and/or disablement device 230 may, in other words, be transitioned to a closed state configuration and provide some indication to the user that this has occurred. In some embodiments, such a first level of engagement may cause the compression structures 238 to exert a first level, degree, or amount of inward pressure upon the fluid reservoir 220. Alternately, during the initial engagement of latch arms 234, 236 the compression structures 238 may not yet be applying inward pressure and the dispensing and/or disablement device may be supported on the BFS vial 210. According to some embodiments, and as depicted in the perspective view of FIG. 2G, a third step may comprise exposing one or more components of the injection assembly 240 in preparation for injection. The injection assembly 240 may comprise, for example, a connector 250 coupled to a needle hub 260 and/or a needle cap 270 that covers a needle 280 that is held by and/or coupled to the needle hub 260. In some embodiments, the needle cap 270 may be removed from the injection assembly 240 to expose the needle 280 during the third step.

According to some embodiments, and as depicted in the perspective view of FIG. 2H and in the cross-sectional side view of FIG. 2I, during use a user may grip the BFS injection system 200 via two (2) opposed grip points “A” and “B” on either side of the BFS assisted dispensing and/or disablement device 230 and advance the needle 280 toward an injection target (e.g., in accordance with a fourth step). In some embodiments, and as depicted with respect to the perspective view of FIG. 2J, FIG. 2K, and the cross-sectional side view of FIG. 2L, a fifth step may comprise the user squeezing (e.g., once the needle 280 has been engaged with the target) the BFS dispensing and/or disablement device 230 (e.g., at the opposing grip points “A” and “B”) to further urge and/or advance the male latch arm 236 into the latching slot 234-2 of the female latch arm 234, thereby further engaging the interior and exterior latching features 234-3, 236-2 thereof (e.g., to a second or subsequent level or stage). According to some embodiments, each consecutive engagement of each respective pair of interior and exterior mating features 234-3, 236-2 may produce an audible and/or otherwise apparent report of second and/or additional levels of engagement and/or mating of the latch arms 234, 236. In some embodiments, each level of engagement may further advance the compression structures 238 to exert second and/or additional levels, degrees, or amounts of inward pressure upon the fluid reservoir 220 until the contents of the fluid reservoir 220 have been properly expelled. In some embodiments, any of the levels or stages of compression may be configured to expel a predetermined amount of fluid form the BFS vial 210. The geometry/sizes of the compression structures 238 combined with the distances between the respective pairs of interior and exterior mating features 234-3, 236-2 may, for example, be specifically configured such that, e.g., a first dose or volume of fluid is displaced from the BFS vial 210 and/or the fluid reservoir 220 thereof in the case that the BFS assistive dispensing and/or disablement device 230 is closed/latched to the first level/stage and a second dose or volume of fluid is displaced from the BFS vial 210 and/or the fluid reservoir 220 thereof in the case that the BFS assistive dispensing and/or disablement device 230 is closed/latched to the second level/stage. According to some embodiments, such as in the case that the fluid reservoir 220 stores one milliliter (1-ml) of fluid, each stage/level may be configured to dispense between one tenth of a milliliter (0.1-ml) and one half of a milliliter (0.5-ml) of the fluid.

According to the exemplary embodiments, use in the manner described and/or shown of the BFS assistive dispensing and/or disablement device 230 may advantageously be employed to easily dispense the contents of fluid reservoir 220 out of the reservoir and into the target, for example through needle 280, without the user being required to directly squeeze or touch the fluid reservoir 220. As the BFS dispensing and/or disablement device 230 necessarily define larger dimensions and contact surfaces than the fluid reservoir 220 (because it is sized to fit around the fluid reservoir 220), the BFS dispensing and/or disablement device 230 provides increased surface area for the user to engage with making squeezing/compression easier for some users. The BFS dispensing and/or disablement device 230 may also provide audible and/or other indexed and/or staged reporting of the advancement level of the squeezing/compression action (for example a visual indication), and/or may become locked in a mated position after delivery of the liquid from the BFS vial 210 so that the BFS vial 210 may not be reused and/or is otherwise readily identifiable as having already been administered.

Fewer or more components 210, 220, 230, 232, 234, 234-1, 234-2, 234-3, 236, 236-1, 236-2, 238, 240, 250, 260, 270, 280 and/or various configurations of the depicted components 210, 220, 230, 232, 234, 234-1, 234-2, 234-3, 236, 236-1, 236-2, 238, 240, 250, 260, 270, 280 may be included in the BFS injection system 200 without deviating from the scope of embodiments described herein. In some embodiments, the components 210, 220, 230, 232, 234, 234-1, 234-2, 234-3, 236, 236-1, 236-2, 238, 240, 250, 260, 270, 280 may be similar in configuration and/or functionality to similarly named and/or numbered components as described herein. In some embodiments, the BFS injection system 200 may comprise a BFS medicament delivery system configured to execute, conduct, and/or facilitate methods described herein such as the method 300 of FIG. 3 herein, or portions thereof.

Referring now to FIG. 3, a flow diagram of a method 300 according to some embodiments is shown. In some embodiments, the method 300 may be performed and/or implemented by a user such as a healthcare worker administering a vaccine or other medicament to a patient. In some embodiments, the method 300 may be conducted by selective manipulation of components of a BFS delivery and/or injection system as described herein.

The process diagrams and flow diagrams described herein do not necessarily imply a fixed order to any depicted actions, steps, and/or procedures, and embodiments may generally be performed in any order that is practicable unless otherwise and specifically noted. While the order of actions, steps, and/or procedures described herein is generally not fixed, in some embodiments, actions, steps, and/or procedures may be specifically performed in the order listed, depicted, and/or described and/or may be performed in response to any previously listed, depicted, and/or described action, step, and/or procedure.

In some embodiments, the method 300 may comprise attaching a dosing collar to a BFS vial, at 302. The dosing collar may comprise, for example, a sleeve, belt, strap, cover and/or other device configured to be coupled around (e.g., to hold) a BFS vial and/or a portion thereof such as a fluid reservoir. According to some embodiments, the dosing collar may be slipped onto the BFS vial and/or coupled thereto. In some embodiments, the dosing collar may be pre-installed (e.g., via an automated assembly process) on the BFS vial so that a user may not need to attached the dosing collar thereto.

According to some embodiments, the method 300 may comprise dispensing a first dose of fluid from the BFS vial by closing the dosing collar to a first stage of closure, at 304. The dosing collar may comprise an indexed closure mechanism, for example, that upon receiving a first compressive/closure force from a user constricts the BFS vial by a first amount as it ratchets to a first stage of closure. In such a manner, for example, the user may selectively administer a first dose of fluid by engaging the dosing collar to transition the dosing collar from an original stage or state (e.g., opened or merely covering the BFS vial) to the first stage wherein a first level of compression is exerted by the dosing collar upon the BFS vial/reservoir. In some embodiments, the first stage of closure and/or first level of compression may be configured to expel a first volume of fluid from the BFS vial/reservoir.

In some embodiments, the method 300 may comprise dispensing a second dose of fluid from the BFS vial by closing the dosing collar to a second stage of closure, at 306. Upon receiving a second and/or continued compressive/closure force from the user, for example, the dosing collar may further constrict the BFS vial by a second amount as it ratchets to a second stage of closure. In such a manner, for example, the user may selectively administer a second dose of fluid by engaging the dosing collar to transition the dosing collar from the first stage of closure to the second stage of closure wherein a second level of compression is exerted by the dosing collar upon the BFS vial/reservoir. In some embodiments, the second stage of closure and/or second level of compression may be configured to expel a second volume of fluid from the BFS vial/reservoir.

According to some embodiments, the method 300 may comprise disabling the BFS vial, at 308. Compression of the dosing collar to the second stage of closure or to a further state of closure may, for example, constrict the BFS vial/reservoir to an extent that all desired volume has been expelled therefrom and may prevent reformation, reconstitution, and/or re-filling of the BFS vial/reservoir. The level of constriction/closure may not be reversible, for example, as the closure indexed closure mechanism may be configured as a one-way ratchet that permits closure/tightening of the dosing vial but mechanically locks the dosing collar, preventing opening thereof. According to some embodiments, a final stage of closure of the dosing vial may also or alternatively pierce the BFS vial/reservoir to render it unsuitable for reuse. An interior surface of the dosing collar may comprise a sharpened and/or piercing element, for example, that is configured to engage the BFS vial/reservoir upon the dosing collar attaining a final stage of closure.

### III. Rules of Interpretation

Throughout the description herein and unless otherwise specified, the following terms may include and/or encompass the example meanings provided. These terms and illustrative example meanings are provided to clarify the language selected to describe embodiments both in the specification and in the appended claims, and accordingly, are not intended to be generally limiting. While not generally limiting and while not limiting for all described embodiments, in some embodiments, the terms are specifically limited to the example definitions and/or examples provided. Other terms are defined throughout the present description.

Neither the Title (set forth at the beginning of the first page of this patent application) nor the Abstract (set forth at the end of this patent application) is to be taken as limiting in any way as the scope of the disclosed invention(s). Headings of sections provided in this patent application are for convenience only, and are not to be taken as limiting the disclosure in any way.

All definitions, as defined and used herein, should be understood to control over dictionary definitions, definitions in documents incorporated by reference, and/or ordinary meanings of the defined terms. The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

The indefinite articles “a” and “an,” as used herein in the specification and in the claims, unless clearly indicated to the contrary, should be understood to mean “at least one” or “one or more”.

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

When an ordinal number (such as “first”, “second”, “third” and so on) is used as an adjective before a term, that ordinal number is used (unless expressly specified otherwise) merely to indicate a particular feature, such as to distinguish that particular feature from another feature that is described by the same term or by a similar term. For example, a “first widget” may be so named merely to distinguish it from, e.g., a “second widget”. Thus, the mere usage of the ordinal numbers “first” and “second” before the term “widget” does not indicate any other relationship between the two widgets, and likewise does not indicate any other characteristics of either or both widgets. For example, the mere usage of the ordinal numbers “first” and “second” before the term “widget” (1) does not indicate that either widget comes before or after any other in order or location; (2) does not indicate that either widget occurs or acts before or after any other in time; and (3) does not indicate that either widget ranks above or below any other, as in importance or quality. In addition, the mere usage of ordinal numbers does not define a numerical limit to the features identified with the ordinal numbers. For example, the mere usage of the ordinal numbers “first” and “second” before the term “widget” does not indicate that there must be no more than two widgets.

An enumerated list of items (which may or may not be numbered) does not imply that any or all of the items are mutually exclusive, unless expressly specified otherwise. Likewise, an enumerated list of items (which may or may not be numbered) does not imply that any or all of the items are comprehensive of any category, unless expressly specified otherwise. For example, the enumerated list “a computer, a laptop, a PDA” does not imply that any or all of the three items of that list are mutually exclusive and does not imply that any or all of the three items of that list are comprehensive of any category.

Numerous embodiments are described in this patent application, and are presented for illustrative purposes only. The described embodiments are not, and are not intended to be, limiting in any sense. The presently disclosed invention(s) are widely applicable to numerous embodiments, as is readily apparent from the disclosure. One of ordinary skill in the art will recognize that the disclosed invention(s) may be practiced with various modifications and alterations, such as structural, logical, software, and electrical modifications. Although particular features of the disclosed invention(s) may be described with reference to one or more particular embodiments and/or drawings, it should be understood that such features are not limited to usage in the one or more particular embodiments or drawings with reference to which they are described, unless expressly specified otherwise.

Devices that are in communication with each other need not be in continuous communication with each other, unless expressly specified otherwise. On the contrary, such devices need only transmit to each other as necessary or desirable, and may actually refrain from exchanging data most of the time. For example, a machine in communication with another machine via the Internet may not transmit data to the other machine for weeks at a time. In addition, devices that are in communication with each other may communicate directly or indirectly through one or more intermediaries.

A description of an embodiment with several components or features does not imply that all or even any of such components and/or features are required. On the contrary, a variety of optional components are described to illustrate the wide variety of possible embodiments of the present invention(s). Unless otherwise specified explicitly, no component and/or feature is essential or required.

Further, although process steps, algorithms or the like may be described in a sequential order, such processes may be configured to work in different orders. In other words, any sequence or order of steps that may be explicitly described does not necessarily indicate a requirement that the steps be performed in that order. The steps of processes described herein may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to the invention, and does not imply that the illustrated process is preferred.

The present disclosure provides, to one of ordinary skill in the art, an enabling description of several embodiments and/or inventions. Some of these embodiments and/or inventions may not be claimed in the present application, but may nevertheless be claimed in one or more continuing applications that claim the benefit of priority of the present application. Applicants intend to file additional applications to pursue patents for subject matter that has been disclosed and enabled but not claimed in the present application.

It will be understood that various modifications can be made to the embodiments of the present disclosure herein without departing from the scope thereof. Therefore, the above description should not be construed as limiting the disclosure, but merely as embodiments thereof. Those skilled in the art will envision other modifications within the scope of the invention as defined by the claims appended hereto.

What is claimed is:

1. A system comprising an accessory for a medical dispensing device, comprising:
an assistive dispensing cover comprising an interior passage sized to fit at least partially around a blow-fill-seal (BFS) vial comprising a collapsible fluid reservoir containing a liquid, an adjustable closure, and at least one compression structure disposed within the interior passage;
wherein the assistive dispensing cover is configured to, in response to receiving a compression force exerted thereon, move the adjustable closure from an open position into a closed position to secure the assistive dispensing cover at least partially around the BFS vial, and
wherein further compressive forces applied to the assistive dispensing cover further advances the adjustable closure until at least one compression structure engages and applies a compressive force to the fluid reservoir.

2. The system of claim 1, further comprising an injection assembly designed to be secured to a distal end of the BFS vial.

3. The system of claim 1, wherein the adjustable closure includes a first latch portion and a second latch portion that are configured to engage each other, and wherein at least one of the first latch portion and the second latch portion are movable upon an application of the compression force.

4. The system of claim 3, wherein the first latch portion and the second latch portion are configured for advancement toward each other in order to move the assistive dispensing cover into the closed position, but are not configured for movement in the opposite direction in order to deter opening of the assistive dispensing cover once in the closed position.

5. The system of claim 3, wherein the first latch portion and the second latch portion are configured for advancement toward each other until the at least one compression structure provides a sufficient force on the fluid reservoir to deliver the liquid from the fluid reservoir.

6. The system of claim 5, wherein the first latch portion and the second latch portion are locked in a mated position after delivery of the liquid so that the dispensing cover may not be removed from the vial without partial destruction of the cover.

7. The system of claim 3, wherein the first latch portion comprises a first mating arm having a plurality of indexed protrusion, and the second latch portion comprises a second mating arm having a plurality of indexed protrusions, and wherein the plurality of indexed protrusions are configured to mate at different stages of engagement between the first mating arm and the second mating arm upon continued application of compression forces.

8. The system of claim 7, wherein the first mating arm includes a slot sized to receive at least a portion of the second mating arm, the plurality of indexed protrusions comprising a plurality of catches supported within the slot, and the plurality of protrusions of the second mating arm comprises a plurality of teeth, and wherein upon insertion of the second mating arm within the first mating arm the plurality of teeth mate with the plurality of catches securing the assistive dispensing cover into the closed position.

9. The system of claim 7, wherein the plurality of indexed protrusions are configured to produce an indication upon achievement of each of the different stages of engagement between the first mating arm and the second mating arm.

10. The system of claim 9, wherein the indication is audible.

11. The system of claim 7, wherein the first mating arm and the second mating arm are configured for advancement toward each other until the at least one compression structure provides a sufficient force on the fluid reservoir to deliver the liquid from the fluid reservoir.

12. The system of claim 11, wherein the first mating arm and the second mating arm are locked in a mated position after delivery of the liquid so that the dispensing cover may not be removed from the vial without partial destruction of the dispensing cover.

13. The system of claim 1, further comprising at least one gripping portion disposed on an outer surface of the assistive dispensing cover.

14. The system of claim 1, wherein the at least one compressive structure comprises a pair of curved compressive structures positioned at opposing sides of the interior passage.

15. A system comprising an accessory for a medical dispensing device, comprising:
a lockable assistive dispensing cover comprising an interior passage sized to fit at least partially around a blow-fill-seal (BFS) vial comprising a collapsible fluid reservoir containing a liquid, an adjustable closure, and at least one compression structure disposed within the interior passage designed to apply pressure to the fluid reservoir upon engagement with the fluid reservoir;
wherein the assistive dispensing cover is configured to, in response to receiving a first compression force, move the adjustable closure from an open position into a closed position to secure the assistive dispensing cover at least partially around the BFS vial, and
wherein additional compression forces applied to the assistive dispensing cover further advances the adjustable closure until at least one compression structure engages the fluid reservoir with a force sufficient to dispense the liquid out of the fluid reservoir.

16. The system of claim 15, further comprising an injection assembly designed to be secured to a distal end of the BFS vial.

17. The system of claim 15, wherein the adjustable closure includes a first mating arm and a second mating arm that are movable between different stages of engagement, with a final stage of engagement being locked in a mated position after dispensing the liquid so that the dispensing cover may not be removed from the vial without partial destruction of the dispensing cover.

18. The system of claim 15, further comprising at least one gripping portion disposed on an outer surface of the assistive dispensing cover.

19. The system of claim 17, wherein the first mating arm includes a slot sized to receive at least a portion of the second mating arm, the first mating arm supporting a plurality of catches within the slot, and the second mating arm supporting a plurality of teeth, and wherein upon insertion of the second mating arm within the first mating arm the plurality of teeth mate with the plurality of catches.

20. The system of claim 17, wherein the adjustable closure is configured to produce an indication upon achievement of each of the different stages of engagement between the first mating arm and the second mating arm.
